(19) United States
(12) Patent Application Publication
(10) Pub. No.: US 2025/0041171 A1
(43) Pub. Date: Feb. 6, 2025

(54) EMULSION COMPOSITIONS CONTAINING CARBOSILOXANE DENDRIMER COMPOUND AND AQUEOUS PHASE FILM FORMER

(71) Applicant: L'OREAL, Paris (FR)

(72) Inventors: Brady ZARKET, Edison, NJ (US); Yixin Yang, Union, NJ (US)

(21) Appl. No.: 18/362,528

(22) Filed: Jul. 31, 2023

(51) Int. Cl. A61K 8/06; A61K 8/67; A61Q 1/00

(52) U.S. Cl. CPC A61K 8/064; A61K 8/675; A61Q 1/00; A61K 2800/43; A61K 2800/48

(57) ABSTRACT

The invention relates to water-in-oil (w/o) emulsion compositions, preferably for skin, including at least one coloring agent, at least one carbosiloxane dendrimer compound and at least one aqueous phase film former, as well as to methods and kits including such emulsion compositions in container(s) or in application.

### FIELD OF THE INVENTION

The present invention relates to water-in-oil (w/o) emulsions (compositions) comprising an aqueous phase dispersed in a continuous oil phase, comprising at least one colorant, at least one carbosiloxane dendrimer compound in the continuous oil phase and at least one aqueous phase film former, as well as to methods and kits comprising such emulsion compositions in container(s) or in application. The compositions have beneficial cosmetic properties including improved wear, sebum resistance, and/or transfer resistance properties. Preferably, the compositions have matte properties as well.

### DISCUSSION OF THE BACKGROUND

At the present time on the market for caring for and making up keratin materials, many products claim staying power throughout the day, withstanding external factors such as sebum, mechanical friction, etc. Long-lasting products for the lips or the face, which can be used at home, are mainly based on synthetic coating polymers in the presence of organic solvents. For example, compositions for making up skin or lips having film forming agent in oil phase are known, where such film forming agents are often silicone resins having the INCI name: Trimethylsiloxysilicate or Polypropylsilsesquioxane.

Besides these long-lasting products, the current trend is towards semi-permanent makeup. Specifically, in recent years, conventional makeup products have met with competition from the market of semi-permanent makeup in professional salons. It is encountered in the sector of makeup for the eyes (semi-permanent mascara, permanent eyelash makeup, eyelash extensions, etc.), for the eyebrows (semi-pigmentation known as micro-blading), for the complexion (freckles, beauty spots or the whole face, glowing or healthy-complexion effect) or for the lips (semi-permanent tattooing). This new trend is driving consumers towards seeking increasingly long staying power for greater practicality (avoiding having to apply and remove makeup daily, healthy complexion effect immediately on waking up, etc.).

The aim of the present invention is to propose compositions which offer excellent staying power of the expected cosmetic effects, notably the color of the makeup on keratin materials (for example, skin and/or lips) which may extend the duration of the cosmetic composition on keratin materials, as well as improve wear of the cosmetic composition on keratin materials and its resistance to external forces such as one or more of mechanical friction, sebum, oil, etc.

In addition, the aim of the present invention is to propose compositions which afford staying power of the expected cosmetic effects, notably the color and/or matte properties of the makeup on keratin materials, combined with a good level of comfort in comparison with conventional systems.

Possibly related patent references include:
U.S. Pat. No. 11,253,462 discloses low-wax compositions containing silicone polymers among other ingredients;
US 2020/0022898 discloses film forming compositions comprising a first anionic or non-ionic non-crosslinking polyacrylate random copolymer and a second cationic non-crosslinking polyamide random copolymer;
U.S. Pat. No. 11,033,478 discloses a self-curling mascara composition containing at least about 7 percent by weight of a non-ionic water-soluble or water dispersible copolymer that includes a cyclic amide monomer, a cyclic amine monomer, and an acrylamide monomer;
U.S. Pat. No. 11,253,462 discloses compositions containing polymethyl methacrylate polymers among other ingredients;
CN 111801092 discloses compositions for impregnating a material, where the compositions contain PEG or polyethyer modified-silicones as well as dendrimer type silioxanes;
FR 3,104,958 discloses compositions containing polyurethane dispersions among other ingredients;
FR 3,110,849 discloses compositions containing polyurethane and/or acrylic dispersions among other ingredients, with little or no fatty material; and
WO 2020/104502 discloses compositions containing at least one specific phenyl silicone oil, a fat-soluble film-forming polymer, a water-soluble film-forming polymer selected from the homopolymers and copolymers of vinylpyrrolidone, and dye materials.
U.S. Pat. No. 11,058,625 discloses compositions containing a dendritic silicone acrylate polymer and a silicone elastomer resin.

There remains a need for improved cosmetic compositions having improved properties with respect to wear of the cosmetic composition on keratin materials and its resistance to external forces such as mechanical friction, sebum and/or oil, as well as compositions which preferably have matte properties.

Accordingly, one aspect of the present invention is a composition which has improved properties with respect to resistance to external forces such as at least one of mechanical friction, sebum, and oil while preferably having matte properties.

### SUMMARY OF THE INVENTION

The present invention relates to water-in-oil (w/o) emulsion compositions comprising an aqueous phase dispersed in a continuous oil phase, comprising: at least one colorant, at least one carbosiloxane dendrimer compound in the continuous oil phase and at least one aqueous phase film former in the aqueous phase. Preferably, the composition is a composition for application to skin. Most preferably, the composition is a foundation. Also preferably, the composition further comprises at least one thickening agent, and/or at least one active agent.

The present invention also relates to methods of treating, caring for and/or making up keratinous material such as skin by applying compositions of the present invention to the keratinous material in an amount sufficient to treat, care for and/or make up the keratinous material. Preferably, the composition is a composition for application to skin. Most preferably, the composition is a foundation. Also preferably, the composition further comprises at least one thickening agent, and/or at least one active agent.

The present invention also relates to methods of making a water-in-oil (w/o) emulsion composition comprising an aqueous phase dispersed in a continuous oil phase, comprising combining at least one carbosiloxane dendrimer compound in an oil phase and at least one aqueous phase film former in an aqueous phase during formation of the emulsion composition. Preferably, the composition is a composition for application to skin. Most preferably, the composition is a foundation. Also preferably, the composition further comprises at least one gelling agent, and/or at least one active agent.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Matte” in compositions as used herein refers to compositions having little to no light reflection. For example, matte compositions can have average gloss properties, measured at 60° in the absence of sebum, of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc. Such measurements can be made by depositing films to be tested onto a substrate (ex. a black scrub panel P121-10N or opacity card BYK No. 2810) using a drawdown bar (for example, of 1 mil, 3 mil, or 6 mil thickness) and an Automatic Drawdown Machine. The films can then be dried and analyzed using a gloss meter (BYK: micro-TRI-gloss) at an angle of such as 60° or 85°.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of phenylated silicone oil” means that an effective amount (that is, a matte-inhibiting effective amount) of phenylated silicone oil is omitted from the composition (that is, about 0% by weight), “substantially free of phenylated silicone oil” means that phenylated silicone oil is present in amounts not greater than 0.25% by weight, and “devoid of phenylated silicone oil” means that phenylated silicone oil is present in amounts not greater than 0.5% by weight, based on the total weight of the composition. The same nomenclature applies for all other ingredients identified throughout the application and in this paragraph such as, for example, (1) mattifying fillers (compositions of the invention which are “free of mattifying fillers,” “substantially free of mattifying fillers,” and “devoid of mattifying fillers,”) and (2) additional film formers other than dendritic silicone acrylate and the at least one aqueous phase film former (compositions of the invention which are “free of additional film formers,” “substantially free of additional film formers,” and “devoid of additional film formers”; or alternatively “free of additional film formers in the oil phase,” “substantially free of additional film formers in the oil phase,” and “devoid of additional film formers in the oil phase”) have meanings consistent with the discussion within this paragraph, even if not specifically discussed for each identified ingredient. Discussed examples of the use of such language such as those in this paragraph are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as skin and evaluating the color of the composition after an extended period of time. For example, the color of a composition may be evaluated immediately following application to keratin materials such as skin and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with keratinous material and having a pleasant color, odor and feel, and which does not cause any unacceptable discomfort (stinging or tautness) liable to discourage a consumer from using the composition. Acceptable PH levels for compositions of the present invention are preferably acidic, that is, less than 7, preferably 6.5 or less, preferably 6.0 or less, preferably 5.5 or less, including all ranges and subranges therebetween such as, for example 3 to 5, 4 to 6, 3 to 4.5, etc. Compositions of the present invention may be in the form of a gel composition.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

The compositions and methods of the present invention can comprise, consist of, or consist essentially of the essential elements and limitations of the invention described herein, as well as any additional or optional ingredients, components, or limitations described herein or otherwise useful. For example, the oil phase film forming system can consist essentially of the at least one carbosiloxane dendrimer compound and/or the aqueous phase film forming system can consist essentially of one aqueous phase film former (e.g., cyclic amide-containing polymer).

For purposes of the present invention, the “basic and novel property” associated with compositions, components and methods which “consist essentially of” identified ingredients or actions is “sebum resistance.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Aqueous Phase Film Former

According to the present invention, compositions including one or more aqueous phase film-forming polymers (“first film-forming polymers” or “first film formers”) that is/are non-ionic water-soluble or water dispersible polymer are provided. For clarity, by “non-ionic water-soluble or water-dispersible” it is meant that the polymer is non-ionic. The polymer is also water-soluble or water-dispersible, particularly to the extent that it can be readily stabilized throughout a vehicle (e.g., water) present in the composition. Preferably, the aqueous phase film-forming polymer(s) also are not emulsifiers: that is, the aqueous phase film-forming polymers preferably do not possess sufficient emulsification properties to stabilize an emulsion to produce a viable emulsion product.

The one or more of these first film-forming polymers are part of a aqueous phase film-forming system. The aqueous phase film forming system can comprise, consist essentially of, or consist of one aqueous phase film former.

Examples of aqueous phase film forming polymer include, but are not limited to, homopolymers and copolymers containing at least one (meth)acrylic acid ((meth)acrylate) monomer and/or at least one vinylpyrrolidone monomer such as polyvinylpyrrolidone, vinyl pyrrolidone/vinyl acetate copolymers, vinyl pyrrolidone/acrylic acid copolymers, vinyl pyrrolidone/acrylate copolymers, alkyl (e.g., butyl)acrylate/hydroxyalkyl (e.g., propyl) dimethicone acrylate copolymers, styrene/acrylates/ammonium methacrylate copolymers, acrylamide/sodium acryloyldimethyltaurate copolymer, etc.; water-soluble polysaccharides such as pectin, unmodified pullulan or cellulose compounds; polyurethanes; latexes; and mixtures thereof.

According to preferred embodiments, the at least one aqueous phase film forming polymer is at least one cyclic amide-containing polymer. Preferably, the at least one cyclic amide-containing polymer also has at least one of cyclic amine and/or acrylamide functionality as well, preferably both of these, although it is possible for the at least one polymer to contain neither cyclic amine nor acrylamide functionality.

Cyclic amide and cyclic amine monomers useful in the first film-forming polymers include those having one or more aromatic or aliphatic ring structures. These rings may have sizes ranging from about 3 to 10, for example, 5 to 8 ring members.

In certain embodiments, monomers useful in forming these first film-forming polymers are polymerizable, ethylenically-unsaturated monomers having a cyclic amine residue or a cyclic amide residue. Accordingly, the cyclic amide monomers of these first film-forming polymers may include cyclic amide residues that are or include heterocyclic ring structures such as lactams and the like. These may include α-Lactam, β-lactam, γ-lactam, δ-lactam, and ε-lactam. In one preferred embodiment, the cyclic amide is a pyrrolidone (a γ-lactam), in particular vinylpyrrolidone

Useful cyclic amine residues may include any of various heterocyclic amines such as azoles, pyrroles, pyrrolidines, carbamates, and the like. In one preferred embodiment, the cyclic amine residue is an imidazole.

In certain embodiments, acrylamide monomers useful in these first film-forming polymers include those having —C3H5NO functional groups. Examples include (meth) acrylamides.

As noted above, at least one cyclic amide-containing polymer preferably has at least one of cyclic amine and acrylamide functionality as well, preferably both, although it is possible that the polymer contains neither functionality. For example, polymer(s) containing cyclic amide residue(s) but not cyclic amine or acrylamide functionality include but are not limited to vinylpyrrolidone homopolymers (polyvinylpyrrolidone or PVP, such as that sold by BASF under the name Luviskol 30 or by Ashland under the name PVP K30L) and vinylpyrrolidone copolymers containing at least one monomer other than vinylpyrrolidone such as, for example, substituted or unsubstituted C₂ (acrylic acid) or C3 (allyl) groups, possibly in the form of esters (for example, acrylates or methacrylates) or ethers, such as vinylpyrrolidone/vinyl acetate (VP/VA) copolymer such as that sold under the name PVP/VA S 630 L by Ashland.

In certain embodiments, the at least one cyclic amide-containing polymer has a weight average molecular weight in a range from about 10,000 daltons to about 1,000,000 daltons, including all ranges and subranges therebetween such as, for example, 50,000 daltons to 500,000 daltons, 75,000 daltons to 300,000 daltons, and 100,000 daltons to 250,000 daltons.

In certain embodiments, the at least one cyclic amide-containing polymer is a copolymer of N-vinyl pyrrolidone, methacrylamide, and N-vinylimidazole.

In certain embodiments, the at least one cyclic amide-containing polymer may be a commercially available variety, such as LUVISET CLEAR AT3, commercially available from BASF of Ludwigshafen, Germany.

According to preferred embodiments, the compositions of the present invention are devoid of, free of, or substantially free of, polyurethanes.

Preferably, the at least one aqueous phase film former is present in the compositions of the present invention in an amount ranging from about 0.5% to about 25% by weight, preferably from about 1% to about 20% by weight, preferably from more than 2% to about 20% by weight, and preferably from about 2.5% to about 15% by weight with respect to the weight of the composition, including all ranges and subranges therebetween such as, for example, 4% to 15%, 2.5% to 15%, 3% to 25%, etc. Preferably, the at least one aqueous phase film former is present in the compositions of the present invention in an amount greater than 2% by weight of the total weight of the composition.

### Carbosiloxane Dendrimer Compound

According to the present invention, compositions including one or more carbosiloxane dendrimer compound(s) (“second film-forming polymers”) are provided. The one or more of these second film-forming polymers are part of the oil phase film-forming system. The oil phase film forming system can comprise, consist essentially of, or consist of the one or more carbosiloxane dendrimer compound(s).

Preferably, the carbosiloxane dendrimer compound(s) are compounds having a backbone and at least one side chain. The term “carbosiloxane dendrimer structure” in the context of the present invention represents a molecular structure with branched groups of high molecular weights, said structure having high regularity in the radial direction starting from the bond to the backbone. Such carbosiloxane dendrimer structures are described in the form of a highly branched siloxane-silylalkylene copolymer in Japanese patent application JP 9-171 154, as well as in U.S. Pat. No. 6,280,748, the entire contents of which are hereby incorporated by reference.

A vinyl polymer according to the invention may contain carbosiloxane dendrimer-based units that may be represented by the following general formula (I):

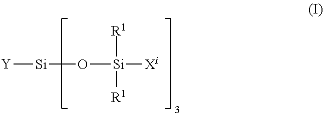

in which:
   R¹ represents an aryl group containing from 5 to 10 carbon atoms or an alkyl group containing from 1 to 10 carbon atoms;
   Xⁱ represents a silylalkyl group which, when i=1, is represented by formula (II):

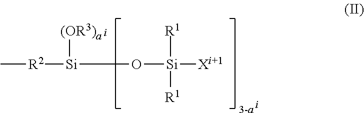

in which:
   R¹ is as defined above in formula (I),
   R² represents an alkylene radical containing from 2 to 10 carbon atoms,
   R³ represents an alkyl group containing from 1 to 10 carbon atoms,
   Xⁱ⁺¹ is chosen from: a hydrogen atom, an alkyl group containing from 1 to 10 carbon atoms, an aryl group containing from 5 to 10 carbon atoms and a silylalkyl group defined above of formula (II) with i=i+1,
   i is an integer from 1 to 10 which represents the generation of said silylalkyl group, and
   aⁱ is an integer from 0 to 3;
   Y represents a radical-polymerizable organic group chosen from:
   organic groups containing a methacrylic group or an acrylic group, said organic groups being represented by the formulae:

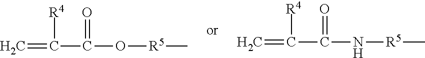

in which:
   R⁴ represents a hydrogen atom or an alkyl group containing from 1 to 10 carbon atoms; and
   R⁵ represents an alkylene group containing from 1 to 10 carbon atoms, such as a methylene group, an ethylene group, a propylene group or a butylene group, methylene and propylene groups being preferred; and
   organic groups containing a styryl group of formula:

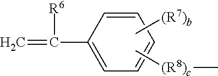

in which:
   R⁶ represents a hydrogen atom or an alkyl group containing from 1 to 10 carbon atoms, such as a methyl group, an ethyl group, a propyl group or a butyl group, the methyl group being preferred:
   R⁷ represents an alkyl group containing from 1 to 10 carbon atoms;
   R⁸ represents an alkylene group containing from 1 to 10 carbon atoms, such as a methylene group, an ethylene group, a propylene group or a butylene group, the ethylene group being preferred;
   b is an integer from 0 to 4; and
   c is 0 or 1, such that, if c is 0, —(R⁸)_{c}— represents a bond.

According to certain embodiments, R¹ may represent an aryl group containing from 5 to 10 carbon atoms or an alkyl group containing from 1 to 10 carbon atoms. The alkyl group may preferably be represented by a methyl group, an ethyl group, a propyl group, a butyl group, a pentyl group, an isopropyl group, an isobutyl group, a cyclopentyl group or a cyclohexyl group. The aryl group may preferably be represented by a phenyl group and a naphthyl group. The methyl and phenyl groups are more particularly preferred, and the methyl group is preferred among all.

According to certain embodiments, R² represents an alkylene group containing from 2 to 10 carbon atoms, in particular a linear alkylene group, such as an ethylene, propylene, butylene or hexylene group; or a branched alkylene group, such as a methylmethylene, methylethylene, 1-methylpentylene or 1,4-dimethylbutylene group.

The ethylene, methylethylene, hexylene, 1-methylpentylene and 1,4-dimethylbutylene groups are preferred among all.

According to one embodiment, R³ is chosen from methyl, ethyl, propyl, butyl and isopropyl groups.

In formula (II), i indicates the number of generations and thus corresponds to the number of repeats of the silylalkyl group.

For example, when the generation number is equal to 1, the carbosiloxane dendrimer may be represented by the general formula shown below, in which Y, R¹, R² and R³ are as defined above, R¹² represents a hydrogen atom or is identical to R¹; a1 is identical to ai. Preferably, the total average number of groups OR3 in a molecule is within the range from 0 to 7.

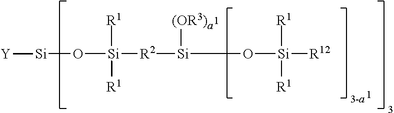

When the generation number is equal to 2, the carbosiloxane dendrimer may be represented by the general formula below, in which Y, R¹, R², R³ and R¹² are the same as defined above; a¹ and a² represent the aⁱ of the indicated generation. Preferably, the total average number of groups OR3 in a molecule is within the range from 0 to 25.

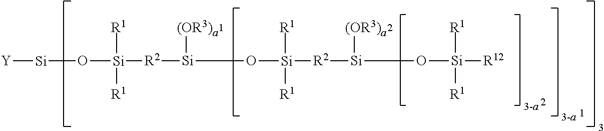

When the generation number is equal to 3, the carbosiloxane dendrimer is represented by the general formula below, in which Y, R¹, R², R³ and R¹² are the same as defined above; a¹, a² and a³ represent the aⁱ of the indicated generation. Preferably, the total average number of groups OR3 in a molecule is within the range from 0 to 79.

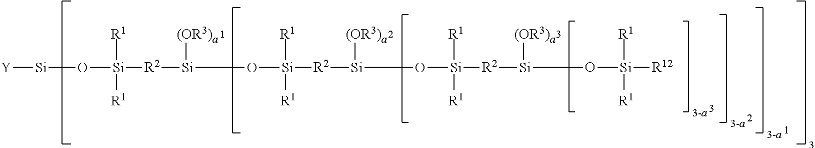

A vinyl polymer containing at least one carbosiloxane dendrimer-based unit has a molecular side chain containing a carbosiloxane dendrimer structure, and may be the product of polymerization of:
(A) from 0 to 99.9 parts by weight of a vinyl monomer; and
(B) from 100 to 0.1 parts by weight of a carbosiloxane dendrimer containing a radical-polymerizable organic group, represented by general formula (I) as defined above.

The monomer of vinyl type that is the component (A) in the vinyl polymer having at least one carbosiloxane dendrimer-based unit is a monomer of vinyl type that contains a radical-polymerizable vinyl group.

There is no particular limitation as regards such a monomer.

The following are examples of this monomer of vinyl type: methyl methacrylate, ethyl methacrylate, n-propyl methacrylate, isopropyl methacrylate or a methacrylate of an analagous lower alkyl; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate or a higher-analogue methacrylate; vinyl acetate, vinyl propionate or a vinyl ester of an analagous lower fatty acid; vinyl caproate, vinyl 2-ethylhexoate, vinyl laurate, vinyl stearate or an ester of a higher fatty acid analogue; styrene, vinyltoluene, benzyl methacrylate, phenoxyethyl methacrylate, vinylpyrrolidone or similar vinylaromatic monomers; methacrylamide, N-methylolmethacrylamide, N-methoxymethylmethacrylamide, isobutoxymethoxymethacrylamide, N,N-dimethylmethacrylamide or similar monomers of vinyl type containing amide groups; hydroxyethyl methacrylate, hydroxypropyl alcohol methacrylate or similar monomers of vinyl type containing hydroxyl groups; acrylic acid, methacrylic acid, itaconic acid, crotonic acid, fumaric acid, maleic acid or similar monomers of vinyl type containing a carboxylic acid group; tetrahydrofurfuryl methacrylate, butoxyethyl methacrylate, ethoxydiethylene glycol methacrylate, polyethylene glycol methacrylate, polypropylene glycol monomethacrylate, hydroxybutyl vinyl ether, cetyl vinyl ether, 2-ethylhexyl vinyl ether or a similar monomer of vinyl type with ether bonds; methacryloxypropyltrimethoxysilane, polydimethylsiloxane containing a methacrylic group on one of its molecular ends, polydimethylsiloxane containing a styryl group on one of its molecular ends, or a similar silicone compound containing unsaturated groups; butadiene; vinyl chloride; vinylidene chloride; methacrylonitrile; dibutyl fumarate; anhydrous maleic acid; anhydrous succinic acid; methacryl glycidyl ether; an organic salt of an amine, an ammonium salt, and an alkali metal salt of methacrylic acid, of itaconic acid, of crotonic acid, of maleic acid or of fumaric acid; a radical-polymerizable unsaturated monomer containing a sulfonic acid group such as a styrenesulfonic acid group; a quaternary ammonium salt derived from methacrylic acid, such as 2-hydroxy-3-methacryloxypropyltrimethylammonium chloride; and a methacrylic acid ester of an alcohol containing a tertiary amine group, such as a methacrylic acid ester of diethylamine.

Multifunctional vinyl monomers may also be used.

The following represent examples of such compounds: trimethylolpropane trimethacrylate, pentaerythrityl trimethacrylate, ethylene glycol dimethacrylate, tetraethylene glycol dimethacrylate, polyethylene glycol dimethacrylate, 1,4-butanediol dimethacrylate, 1,6-hexanediol dimethacrylate, neopentyl glycol dimethacrylate, trimethylolpropanetrioxyethyl methacrylate, tris(2-hydroxyethyl) isocyanurate dimethacrylate, tris(2-hydroxyethyl) isocyanurate trimethacrylate, polydimethylsiloxane capped with styryl groups containing divinylbenzene groups on both ends, or similar silicone compounds containing unsaturated groups.

A carbosiloxane dendrimer, which is the component (B), may be represented by formula (I) as defined above.

The following represent the preferred examples of group Y of formula (I): an acryloxymethyl group, a 3-acryloxypropyl group, a methacryloxymethyl group, a 3-methacryloxypropyl group, a 4-vinylphenyl group, a 3-vinylphenyl group, a 4-(2-propenyl)phenyl group, a 3-(2-propenyl)phenyl group, a 2-(4-vinylphenyl)ethyl group, a 2-(3-vinylphenyl)ethyl group, a vinyl group, an allyl group, a methallyl group and a 5-hexenyl group.

A carbosiloxane dendrimer according to the present invention can be represented by the formulae having the average structures below:

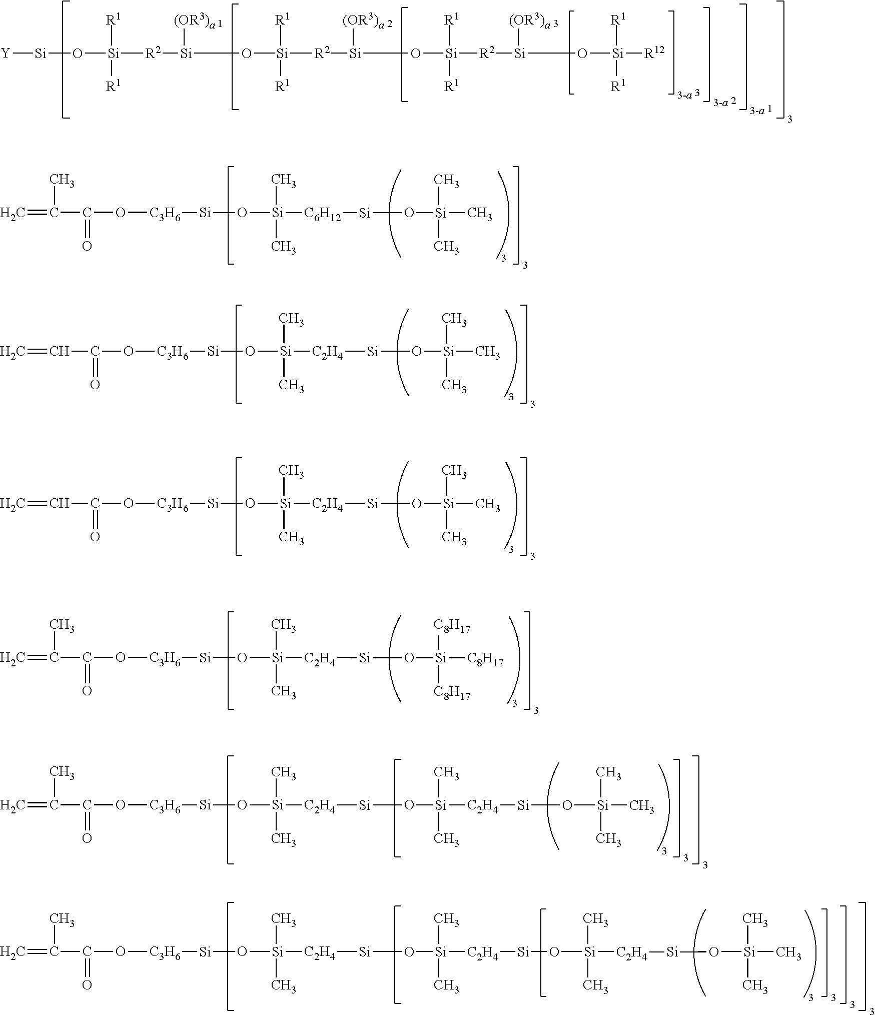

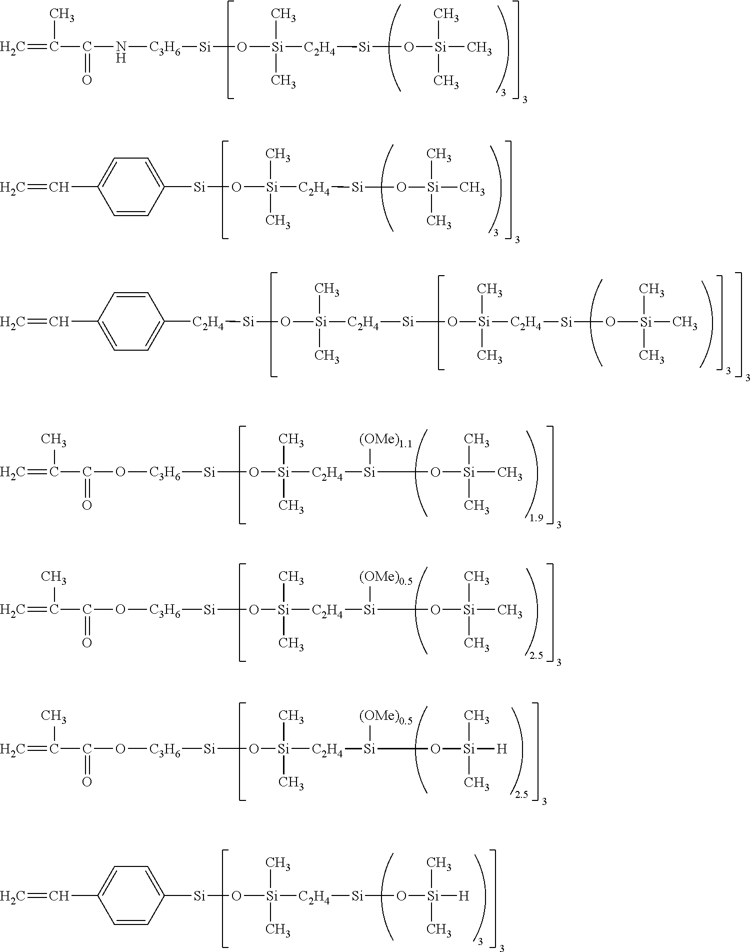

Thus, according to one embodiment, the carbosiloxane dendrimer of the composition according to the present invention is represented by the following formula:

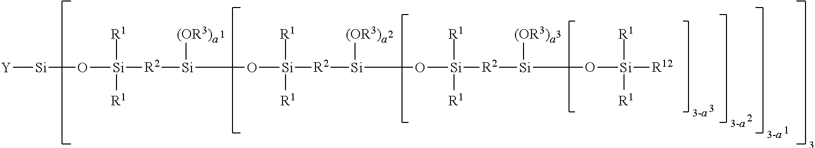

in which:
   Y, R¹, R² and R³ are as defined in formulae (I) and (II) above;
   a¹, a² and a³ correspond to the definition of aⁱ according to formula (II); and
   R¹² is H, an aryl group containing from 5 to 10 carbon atoms or an alkyl group containing from 1 to 10 carbon atoms.

According to one embodiment, the carbosiloxane dendrimer of the composition according to the present invention is represented by one of the following formulae:

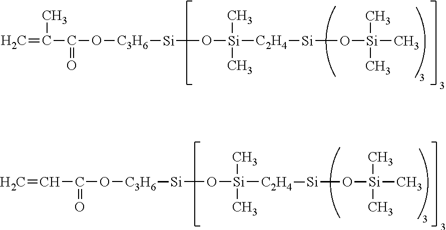

The vinyl polymer comprising the carbosiloxane dendrimer according to the invention may be manufactured according to the process for manufacturing a branched silalkylene siloxane described in Japanese patent application Hei 9-171 154.

For example, it may be produced by subjecting an organosilicon compound containing a hydrogen atom linked to a silicon atom, represented by the following general formula (IV):

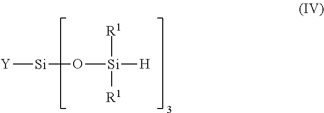

R¹ being as defined above in formula (I),
and an organosilicon compound containing an alkenyl group, to a hydrosilylation reaction.

In the above formula, the organosilicon compound may be represented by 3-methacryloxypropyltris(dimethylsiloxy) silane, 3-acryloxypropyltris(dimethylsiloxy) silane and 4-vinylphenyltris(dimethylsiloxy) silane. The organosilicon compound that contains an alkenyl group may be represented by vinyltris(trimethylsiloxy) silane, vinyltris(dimethylphenylsiloxy) silane, and 5-hexenyltris(trimethylsiloxy) silane.

The hydrosilylation reaction is performed in the presence of a chloroplatinic acid, a complex of vinylsiloxane and of platinum, or a similar transition metal catalyst.

A vinyl polymer containing at least one carbosiloxane dendrimer-based unit may be chosen from polymers such that the carbosiloxane dendrimer-based unit is a carbosiloxane dendritic structure represented by formula (III):

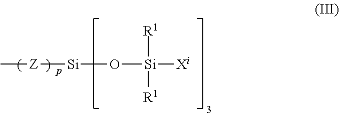

in which Z is a divalent organic group, “p” is 0 or 1, R¹ is as defined above in formula (IV) and Xi is a silylalkyl group represented by formula (II) as defined above.

In a vinyl polymer containing at least one carbosiloxane dendrimer-based unit, the polymerization ratio between the components (A) and (B), in terms of the weight ratio between (A) and (B), is within a range from 0/100 to 99.9/0.1, or even from 0.1/99.9 to 99.9/0.1 and preferably within a range from 1/99 to 99/1. A ratio between the components (A) and (B) of 0/100 means that the compound becomes a homopolymer of component (B).

A vinyl polymer containing at least one carbosiloxane dendrimer-based unit may be obtained by copolymerization of the components (A) and (B), or by polymerization of the component (B) alone.

The polymerization may be a free-radical polymerization or an ionic polymerization, but free-radical polymerization is preferred.

The polymerization may be performed by bringing about a reaction between the components (A) and (B) in a solution for a period of from 3 to 20 hours in the presence of a radical initiator at a temperature of from 50° C. to 150° C.

A suitable solvent for this purpose is hexane, octane, decane, cyclohexane or a similar aliphatic hydrocarbon; benzene, toluene, xylene or a similar aromatic hydrocarbon; diethyl ether, dibutyl ether, tetrahydrofuran, dioxane or ethers; acetone, methyl ethyl ketone, methyl isobutyl ketone, diisobutyl ketone or similar ketones; methyl acetate, ethyl acetate, butyl acetate, isobutyl acetate or similar esters; methanol, ethanol, isopropanol, butanol or similar alcohols; octamethylcyclotetrasiloxane, decamethylcyclopentasiloxane, hexamethyldisiloxane, octamethyltrisiloxane or a similar organosiloxane oligomer.

A radical initiator may be any compound known in the art for standard free-radical polymerization reactions. The specific examples of such radical initiators are 2,2′-azobis(isobutyronitrile), 2,2′-azobis(2-methylbutyronitrile), 2,2′-azobis(2,4-dimethylvaleronitrile) or similar compounds of azobis type; benzoyl peroxide, lauroyl peroxide, tert-butyl peroxybenzoate, tert-butyl peroxy-2-ethylhexanoate or a similar organic peroxide. These radical initiators may be used alone or in a combination of two or more. The radical initiators may be used in an amount of from 0.1 to 5 parts by weight per 100 parts by weight of the components (A) and (B). A chain-transfer agent may be added. The chain-transfer agent may be 2-mercaptoethanol, butyl mercaptan, n-dodecyl mercaptan, 3-mercaptopropyltrimethoxysilane, a polydimethylsiloxane containing a mercaptopropyl group or a similar compound of mercapto type; methylene chloride, chloroform, carbon tetrachloride, butyl bromide, 3-chloropropyltrimethoxysilane or a similar halogenated compound.

In the manufacture of the polymer of vinyl type, after the polymerization, the unreacted residual vinyl monomer may be removed under conditions of heating under vacuum.

To facilitate the preparation of starting material for cosmetic products, the number-average molecular weight of the vinyl polymer containing a carbosiloxane dendrimer may be chosen within the range between 3000 and 2 000 000 and preferably between 5000 and 800 000. It may be a liquid, a gum, a paste, a solid, a powder, or any other form. The preferred forms are solutions consisting of the dilution of a dispersion or of a powder in solvents.

The vinyl polymer may be a dispersion of a polymer of vinyl type having a carbosiloxane dendrimer structure in its side molecular chain, in a liquid such as a silicone oil, an organic oil, an alcohol or water.

The silicone oil may be a dimethylpolysiloxane having the two molecular ends capped with trimethylsiloxy groups, a copolymer of methylphenylsiloxane and of dimethylsiloxane having the two molecular ends capped with trimethylsiloxy groups, a copolymer of methyl-3,3,3-trifluoropropylsiloxane and of dimethylsiloxane having the two molecular ends capped with trimethylsiloxy groups, or similar unreactive linear silicone oils, and also hexamethylcyclotrisiloxane, octamethylcyclotetrasiloxane, decamethylcyclopentasiloxane, dodecamethylcyclohexasiloxane or a similar cyclic compound. In addition to the unreactive silicone oils, modified polysiloxanes containing functional groups such as silanol groups, amino groups and polyether groups on the ends or within the molecular side chains may be used.

The organic oils may be isododecane, liquid paraffin, isoparaffin, hexyl laurate, isopropyl myristate, myristyl myristate, cetyl myristate, 2-octyldodecyl myristate; isopropyl palmitate, 2-ethylhexyl palmitate, butyl stearate, decyl oleate, 2-octyldodecyl oleate, myristyl lactate, cetyl lactate, lanolin acetate, stearyl alcohol, cetostearyl alcohol, oleyl alcohol, avocado oil, almond oil, olive oil, cocoa oil, jojoba oil, gum oil, sunflower oil, soybean oil, camellia oil, squalane, castor oil, cottonseed oil, coconut oil, egg yolk oil, polypropylene glycol monooleate, neopentyl glycol 2 ethylhexanoate or a similar glycol ester oil; triglyceryl isostearate, the triglyceride of a fatty acid of coconut oil, or a similar oil of a polyhydric alcohol ester; polyoxyethylene lauryl ether, polyoxypropylene cetyl ether or a similar polyoxyalkylene ether.

The alcohol may be any type that is suitable for use in combination with a cosmetic product starting material. For example, it may be methanol, ethanol, butanol, isopropanol or similar lower alcohols.

A solution or a dispersion of the alcohol should have a viscosity within the range from 10 to 10⁹ mPa at 25° C. To improve the sensory use properties in a cosmetic product, the viscosity should be within the range from 100 to 5×10⁸ mPa·s.

The solutions and dispersions may be readily prepared by mixing a vinyl polymer containing at least one carbosiloxane dendrimer-based unit with a silicone oil, an organic oil, an alcohol or water. The liquids may be present in the polymerization step. In this case, the unreacted residual vinyl monomer should be completely removed by heat treatment of the solution or dispersion under atmospheric pressure or reduced pressure.

In the case of a dispersion, the dispersity of the polymer of vinyl type may be improved by adding a surfactant.

Such an agent may be hexylbenzenesulfonic acid, octylbenzenesulfonic acid, decylbenzenesulfonic acid, dodecylbenzenesulfonic acid, cetylbenzenesulfonic acid, myristylbenzenesulfonic acid or anionic surfactants of the sodium salts of these acids; octyltrimethylammonium hydroxide, dodecyltrimethylammonium hydroxide, hexadecyltrimethylammonium hydroxide, octyldimethylbenzylammonium hydroxide, decyldimethylbenzylammonium hydroxide, dioctadecyldimethylammonium hydroxide, beef tallow-trimethylammonium hydroxide, coconut oil-trimethylammonium hydroxide, or a similar cationic surfactant; a polyoxyalkylene alkyl ether, a polyoxyalkylenealkylphenol, a polyoxyalkylene alkyl ester, the sorbitol ester of polyoxyalkylene, polyethylene glycol, polypropylene glycol, an ethylene oxide additive of diethylene glycol trimethylnonanol, and non-ionic surfactants of polyester type, and also mixtures.

In the dispersion, a mean particle diameter of the polymer of vinyl type may be within a range of between 0.001 and 100 microns and preferably between 0.01 and 50 microns. The reason for this is that, outside the recommended range, a cosmetic product mixed with the emulsion will not have a nice enough feel on the lips or to the touch, nor sufficient spreading properties nor a pleasant feel.

A vinyl polymer contained in the dispersion or the solution may have a concentration within a range of between 0.1% and 95% by weight and preferably between 5% and 85% by weight. However, to facilitate the handling and the preparation of the mixture, the range should preferably be between 10% and 75% by weight.

A vinyl polymer that is suitable for use in the invention may also be one of the polymers described in the examples of patent application EP 0 963 751.

According to one preferred embodiment, a vinyl polymer grafted with a carbosiloxane dendrimer may be the product of polymerization of:
(A1) from 0 to 99.9 parts by weight of one or more acrylate or methacrylate monomer(s); and
(B1) from 100 to 0.1 parts by weight of an acrylate or methacrylate monomer of a tris [tri (trimethylsiloxy) silylethyldimethylsiloxy]silylpropyl carbosiloxane dendrimer.

The monomers (A1) and (B1) correspond respectively to specific monomers (A) and (B).

According to one embodiment, a vinyl polymer containing at least one carbosiloxane dendrimer-based unit may comprise a tris [tri (trimethylsiloxy) silylethyldimethylsiloxy]silylpropyl carbosiloxane dendrimer-based unit corresponding to one of the formulae:

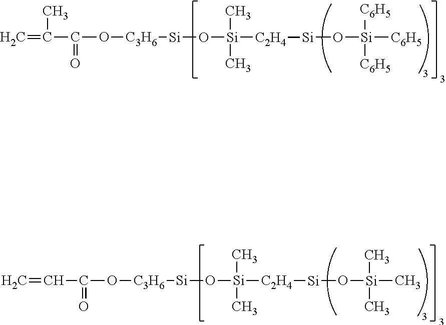

According to one preferred mode, a vinyl polymer containing at least one carbosiloxane dendrimer-based unit used in the invention comprises at least one butyl acrylate monomer.

According to one embodiment, a vinyl polymer may also comprise at least one fluoro organic group.

Structures in which the polymerized vinyl units constitute the backbone and carbosiloxane dendritic structures and also fluoro organic groups are attached to side chains are particularly preferred.

The fluoro organic groups may be obtained by replacing with fluorine atoms all or some of the hydrogen atoms of methyl, ethyl, propyl, isopropyl, butyl, isobutyl, tert-butyl, pentyl, neopentyl, hexyl, cyclohexyl, heptyl, octyl, nonyl, decyl, undecyl, dodecyl, tridecyl, tetradecyl, hexadecyl and octadecyl groups and other alkyl groups of 1 to 20 carbon atoms, and also alkyloxyalkylene groups of 6 to 22 carbon atoms.

The groups represented by the formula —(CH₂)ₓ—(CF₂)_{y}—R¹³ are suggested as examples of fluoroalkyl groups obtained by substituting fluorine atoms for hydrogen atoms of alkyl groups. In the formula, the index “x” is 0, 1, 2 or 3, and “y” is an integer from 1 to 20. R¹³ is an atom or a group chosen from a hydrogen atom, a fluorine atom, —(CH(CF₃)₂— or CF(CF₃)₂. Such fluorine-substituted alkyl groups are exemplified by linear or branched polyfluoroalkyl or perfluoroalkyl groups represented by the formulae shown below: —CF₃, —C₂F₅, -nC₃F₇, —CF(CF₃)₂, nC₄F₉, CF₂CF(CF₃)₂, -nC₅F₁₁, -nC₆F₁₃,-nC₈F₁₇, CH₂CF₃, —(CH(CF₃)₂, CH₂CH(CF₃)₂—CH₂ (CF₂)₂F, —CH₂ (CF₂)₃F, —CH₂ (CF₂)₄F, CH₂ (CF₂)₆F, CH₂ (CF₂)₈F, —CH₂CH₂CF₃, —CH₂CH₂ (CF₂)₂F, —CH₂CH₂ (CF₂)₃F, —CH₂CH₂ (CF₂)₄F, —CH₂CH₂ (CF₂)₆F, —CH₂CH₂ (CF₂)₈F, —CH₂CH₂ (CF₂)₁₀F, —CH₂CH₂ (CF₂)₁₂F, CH₂CH₂ (CF₂)₁₄F, —CH₂CH₂ (CF₂)₁₆F, —CH₂CH₂CH₂CF₃, —CH₂CH₂CH₂ (CF₂)₂F, —CH₂CH₂CH₂ (CF₂)₂H, —CH₂ (CF₂)₄H and —CH₂CH₂ (CF₂)₃H.

The groups represented by —CH₂CH₂—(CF₂)ₘ—CFR¹⁴—[OCF₂CF(CF₃)]ₙ—OC₃F₇ are suggested as fluoroalkyloxyfluoroalkylene groups obtained by substituting fluorine atoms for hydrogen atoms of alkyloxyalkylene groups. In the formula, the index “m” is 0 or 1, “n” is 0, 1, 2, 3, 4 or 5, and R¹⁴ is a fluorine atom or CF₃. Such fluoroalkyloxyfluoroalkylene groups are exemplified by the perfluoroalkyloxyfluoroalkylene groups represented by the formulae shown below: —CH₂CH₂CF(CF₃)—[OCF₂CF(CF₃)]ₙ—OC₃F₇, —CH₂CH₂CF₂CF₂—[OCF₂CF(CF₃)]ₙ—OC₃F₇.

The number-average molecular weight of the vinyl polymer used in the present invention may be between 3000 and 2 000 000 and more preferably between 5000 and 800 000.

This type of fluorinated vinyl polymer may be obtained by addition:
of a vinyl monomer (M2) without a fluoro organic group,
on a vinyl monomer (M1) containing fluoro organic groups, and
a carbosiloxane dendrimer (B) as defined above, of general formula (I) as defined above,
by subjecting them to a copolymerization.

Thus, according to one embodiment, a composition of the invention may comprise a vinyl polymer which has at least one carbosiloxane dendrimer-based unit and which results from the copolymerization of a vinyl monomer (M1) as defined above, optionally of a vinyl monomer (M2) as defined above, and of a carbosiloxane dendrimer (B) as defined above,

said vinyl polymer having a copolymerization ratio between the monomer (M1) and the monomer (M2) of 0.1 to 100:99.9 to 0% by weight, and a copolymerization ratio between the sum of the monomers (M1) and (M2) and the monomer (B) of 0.1 to 99.9:99.9 to 0.1% by weight.

The vinyl monomers (M1) containing fluoro organic groups in the molecule are preferably monomers represented by the general formula:

(CH₂)═CR¹⁵COOR^{f}.

In this formula, R¹⁵ is a hydrogen atom or a methyl group and R^{f} is a fluoro organic group exemplified by the fluoroalkyl and fluoroalkyloxyfluoroalkylene groups described above. The compounds represented by the formulae presented below are suggested as specific examples of the component (M1). In the formulae presented below, “z” is an integer from 1 to 4.
CH₂═CCH₃COO—CF₃, CH₂═CCH₃COO—C₂F₅, CH₂═CCH₃COO-nC₃F₇,
CH₂═CCH₃COO—CF(CF₃)₂, CH₂═CCH₃COO-nC₄F₉,
CH₂═CCH₃COO—CF(CF₃)₂, CH₂═CCH₃COO-nC₅F₁₁,
CH₂—CCH3COO-nC6F13, CH2=CCH3OO-nC8F17 CH2=CCH3COO—CH2CF3,
CH2=CCH3COO—CH(CF3)2, CH2=CCH3COO—CH2CH(CF3)2,
CH₂═CCH₃COO—CH2 (CF2)2F, CH2=CCH3COO—CH2 (CF2)2F,
CH2-CCH3COO—CH2 (CF2)4F, CH2=CCH3COO—CH2 (CF2)6F,
CH2=CCH3COO—CH2 (CF2)8F, CH2=CCH3COO—CH2CH2CF3,
CH2=CCH3COO—CH2CH2 (CF2)2F, CH2=CCH3COO—CH2CH2 (CF2)3F,
CH2=CCH3COO—CH2CH2 (CF2)4F, CH2=CCH3COO—CH2CH2 (CF2)6F,
CH2=CCH3COO—CH2CH2 (CF2)8F, CH2=CCH3COO—CH2CH2 (CF2)10F,
CH2=CCH3COO—CH2CH2 (CF2)12F, CH2=CCH3COO—CH2CH2 (CF2)14F,
CH2=CCH3COO—CH2-CH2-(CF2)16F, CH2=CCH3COO—CH2CH2CH2CF3,
CH2=CCH3COO—CH2CH2CH2 (CF2)2H, CH2=CCH3COO—CH2 (CF2)2H,
CH2=CCH3COO—CH2 (CF2)4F, CH2=CCH3COO—CH2 (CF2)3F,
CH2=CCH3COO—CH2CH2CF3CF2-[OCF2-CF(CF3)]z-OC3F7,
CH2=CCH3COO—CH2CH2CF2CF2-[OCF2-CF(CF3)]z-OC3F7,
CH2=CHCOO—CF3, CH2=CHCOO—C2F5, CH2=CHCOO-nC3F7,
CH2=CHCOO-nC3F2, CH2=CHCOO-nC4F9, CH2=CHCOO—CH2CF3,
CH2=CHCOO-nC5F11, CH2=CHCOO-nC6F13, CH2=CHCOO-nC8F17,
CH2-CHCOO—CH2CF3, CH2=CHCOO—CH(CF3)2, CH2=CHCOO—CH2CH(CF3)2,
CH2=CHCOO—CH2 (CF2)2F, CH2=CHCOO—CH2 (CF2)3F,
CH2-CHCOO—CH2 (CF2)4F, CH2=CHCOO—CH2 (CF2)6F,
CH2=CHCOO—CH2 (CF2)8F, CH2=CHCOO—CH2CH2CF3,
CH2=CHCOO—CH2CH2 (CF2)2F, CH2=CHCOO—CH2CH2 (CF2)3F,
CH2=CHCOO—CH2CH2 (CF2)4F, CH2=CHCOO—CH2CH2 (CF2)6F,
CH2=CHCOO—CH2CH2 (CF2)8F, CH2=HCOO—CH2CH2 (CF2)10F,
CH2-CHCOO—CH2CH2-(CF2)12F, CH2=CHCOO—CH2CH2 (CF2)14F,
CH2=CHCOO—CH2CH2 (CF2)16F, CH2-CHCOO—CH2CH2CH2CF3,
CH2=CCH3COO—CH2CH2CH2 (CF2)2H, CH2=CCH3COO—CH2 (CF2)2H,
CH2=CCH3COO—CH2 (CF2)4F, CH2=CCH3COO—CH2 (CF2)3F,
CH2=CCH3COO—CH2CH2CF3CF2-[OCF2-CF(CF3)]z-OC3F7,
CH2=CCH3COO—CH2CH2CF2CF2-[OCF2-CF(CF3)]z-OC3F7,
CH2=CHCOO—CF3, CH2=CHCOO—C2F5, CH2=CHCOO-nC3F7,
CH2=CHCOO-nC3F2, CH2=CHCOO-nC4F9, CH2=CHCOO—CH2CF3,
CH2-CHCOO-nC5F11, CH2=CHCOO-nC6F13, CH2=CHCOO-nC8F17,
CH2=CHCOO—CH2CF3, CH2=CHCOO—CH(CF3)2, CH2=CHCOO—CH2CH(CF3)2,
CH2=CHCOO—CH2 (CF2)2F, CH2=CHCOO—CH2 (CF2)3F,
CH2=CHCOO—CH2 (CF2)4F, CH2=CHCOO—CH2 (CF2)6F,
CH2=CHCOO—CH2 (CF2)8F, CH2=CHCOO—CH2CH2CF3,
CH2-CHCOO—CH2CH2 (CF2)2F, CH2=CHCOO—CH2CH2 (CF2)3F,
CH2=CHCOO—CH2CH2 (CF2)4F, CH2=CHCOO—CH2CH2 (CF2)6F,
CH2-CHCOO—CH2CH2 (CF2)8F, CH2=HCOO—CH2CH2 (CF2)10F,
CH2-CHCOO—CH2CH2-(CF2)12F, CH2=CHCOO—CH2CH2 (CF2)14F,
CH2=CHCOO—CH2CH2 (CF2)16F, CH2=CHCOO—CH2CH2CH2CF3,
CH₂═CHCOO—CH2CH2CH2 (CF2)2F, CH2=CHCOO—CH2CH2CH2 (CF)2H,
CH2=CHCOO—CH2 (CF2)4H, CH2=CHCOO—CH2CH2 (CF2)3H,
CH2-CHCOO—CH2CH2CF(CF3)-, [OCF2-CF(CF3)]Z—OC3F7,
CH2=CHCOO—CH2CH2CF2CF2 (CF3)-[OCF2-CF(CF3)]2-OC3F7.

Among these, the vinyl polymers represented by the formulae presented below are preferable:
CH₂═CHCOO—CH2CH2 (CF2)6F, CH2=CHCOO—CH2CH2 (CF2)8F,
CH2=CCH3COO—CH2CH2 (CF2)6F, CH2=CCH3COO—CH2CH2 (CF2)8F,
CH2=CCH3COO—CH2CH2 (CF2)6F, CH2=CCH3COO—CH2CH2 (CF2)8F,
CH2=CHCOO—CH2CF3, CH2=CCH3COO—CH2CF3.

The vinyl polymers represented by the formulae presented below are particularly preferable:
CH₂═CHCOO—CH₂CF₃, CH₂═CCHCOO—CH₂CF₃.

The vinyl monomers (M2) not containing any organofluorine groups in the molecule may be any monomers containing radical-polymerizable vinyl groups which are exemplified, for example, by methyl acrylate, methyl methacrylate, ethyl acrylate, ethyl methacrylate, n-propyl acrylate, n-propyl methacrylate, isopropyl acrylate, isopropyl methacrylate, and other lower alkyl acrylates or methacrylates; glycidyl acrylate, glycidyl methacrylate; n-butyl acrylate, n-butyl methacrylate, isobutyl acrylate, isobutyl methacrylate, tert-butyl acrylate, tert-butyl methacrylate, n-hexyl acrylate, n-hexyl methacrylate, cyclohexyl acrylate, cyclohexyl methacrylate, 2-ethylhexyl acrylate, 2-ethylhexyl methacrylate, octyl acrylate, octyl methacrylate, lauryl acrylate, lauryl methacrylate, stearyl acrylate, stearyl methacrylate, and other higher acrylates and methacrylates; vinyl acetate, vinyl propionate and other lower fatty acid vinyl esters; vinyl butyrate, vinyl caproate, vinyl 2-ethylhexanoate, vinyl laurate, vinyl stearate, and other higher fatty acid esters; styrene, vinyltoluene, benzyl acrylate, benzyl methacrylate, phenoxyethyl acrylate, phenoxyethyl methacrylate, vinylpyrrolidone, and other vinyl aromatic monomers; dimethylaminoethyl acrylate, dimethylaminoethyl methacrylate, diethylaminoethyl acrylate, diethylaminoethyl methacrylate, and other aminovinyl monomers, acrylamide, methacrylamide, N-methylolacrylamide, N-methylolmethacrylamide, N-methoxymethylacrylamide, N-methoxymethylmethacrylamide, isobutoxymethoxyacrylamide, isobutoxymethoxymethacrylamide, N,N-dimethylacrylamide, N,N-dimethylmethacrylamide, and other vinylamide monomers; hydroxyethyl acrylate, hydroxyethyl methacrylate, acrylic acid hydroxypropyl alcohol, methacrylic acid hydroxypropyl alcohol, and other hydroxyvinyl monomers; acrylic acid, methacrylic acid, itaconic acid, crotonic acid, fumaric acid, maleic acid, and other vinylcarboxylic acid monomers; tetrahydrofurfuryl acrylate, tetrahydrofurfuryl methacrylate, butoxyethyl acrylate, butoxyethyl methacrylate, ethoxydiethylene glycol acrylate, ethoxydiethylene glycol methacrylate, polyethylene glycol acrylate, polyethylene glycol methacrylate, polypropylene glycol monoacrylate, polypropylene glycol monomethacrylate, hydroxybutyl vinyl ether, cetyl vinyl ether, 2-ethylhexyl vinyl ether, and other vinyl monomers containing an ether bond; acryloxypropyltrimethoxysilane, methacryloxypropyltrimethoxysilane, polydimethylsiloxanes containing acryl or methacryl groups at one of the ends, polydimethylsiloxanes containing alkenylaryl groups at one of the ends and other silicone compounds containing unsaturated groups; butadiene; vinyl chloride; vinylidene chloride, acrylonitrile, methacrylonitrile; dibutyl fumarate; maleic anhydride; dodecylsuccinic anhydride; acryl glycidyl ether, methacryl glycidyl ether, 3,4-epoxycyclohexylmethyl acrylate, 3,4-epoxycyclohexylmethyl methacrylate, alkali metal salts, ammonium salts and organic amine salts of acrylic acid, of methacrylic acid, of itaconic acid, of crotonic acid, of fumaric acid, of maleic acid and of other radical-polymerizable unsaturated carboxylic acids, radical-polymerizable unsaturated monomers containing sulfonic acid groups, such as styrene sulfonic acid and also the alkali metal salts thereof, the ammonium salts thereof and the organic amine salts thereof; the quaternary ammonium salts derived from acrylic acid or methacrylic acid, such as 2-hydroxy-3-methacryloxypropyltrimethylammonium chloride, methacrylic acid esters of a tertiary amine alcohol, such as the diethylamine ester of methacrylic acid and quaternary ammonium salts thereof.

In addition, it is also possible to use as vinyl monomers (M2) the polyfunctional vinyl monomers illustrated, for example, by trimethylolpropane triacrylate, trimethylolpropane trimethacrylate, pentaerythrityl triacrylate, pentaerythrityl trimethacrylate, ethylene glycol diacrylate, ethylene glycol dimethacrylate, tetraethylene glycol diacrylate, tetraethylene glycol dimethacrylate, polyethylene glycol diacrylate, polyethylene glycol dimethacrylate, 1,4-butanediol diacrylate, 1,4-butanediol dimethacrylate, 1,6-hexanediol diacrylate, 1,6-hexanediol dimethacrylate, neopentyl glycol diacrylate, neopentyl glycol dimethacrylate, trimethylolpropanetrioxyethyl acrylate, trimethylolpropanetrioxyethyl methacrylate, tris(2-hydroxyethyl) isocyanurate diacrylate, tris(2-hydroxyethyl) isocyanurate dimethacrylate, tris(2-hydroxyethyl) isocyanurate triacrylate, tris(2-hydroxyethyl) isocyanurate trimethacrylate, polydimethylsiloxane in which the two ends of the molecular chain are blocked with alkenylaryl groups, and other silicone compounds containing unsaturated groups.

As regards the ratio mentioned above in which (M1) and (M2) are copolymerized, the weight ratio between (M1) and (M2) is preferably within the range 1:99 to 100:0.

Y can be chosen, for example, from organic groups containing acrylic or methacrylic groups, organic groups containing an alkenylaryl group, or alkenyl groups containing from 2 to 10 carbon atoms.

The organic groups containing acrylic or methacrylic groups and the alkenylaryl groups are as defined above.

Among the compounds (B), mention may, for example, be made of the following compounds:

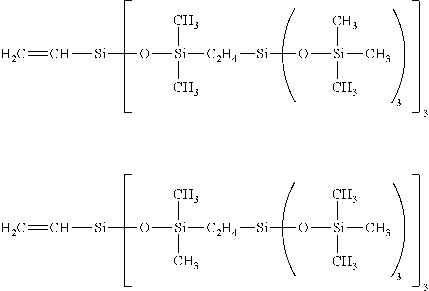

The carbosiloxane dendrimers (B) may be prepared using the process for preparing siloxane/silalkylene branched copolymers described in document EP 1 055 674.

For example, they may be prepared by subjecting organic alkenyl silicone compounds and silicone compounds comprising hydrogen atoms bonded to the silicon, represented by formula (IV) as defined above, to a hydrosilylation reaction.

The copolymerization ratio (by weight) between the monomer (B) and the monomers (M1) and (M2) is preferably within the range of 1:99 to 99:1 and even more preferably within the range of 5:95 to 95:5.

Amino groups may be introduced into the side chains of the vinyl polymer using, included in the component (M2), vinyl monomers containing amino groups, such as dimethylaminoethyl acrylate, dimethylaminoethyl methacrylate, diethylaminoethyl acrylate and diethylaminoethyl methacrylate, followed by performing a modification with potassium acetate monochloride, ammonium acetate monochloride, the aminomethylpropanol salt of monochloroacetic acid, the triethanolamine salt of monobromoacetic acid, sodium monochloropropionate, and other alkali metal salts of halogenated fatty acids; otherwise, carboxylic acid groups may be introduced into the side chains of the vinyl polymer using, included in the component (M2), vinyl monomers containing carboxylic acids, such as acrylic acid, methacrylic acid, itaconic acid, crotonic acid, fumaric acid and maleic acid, and the like, followed by neutralizing the product with triethylamine, diethylamine, triethanolamine and other amines.

A fluorinated vinyl polymer may be one of the polymers described in the examples of patent application WO 03/045 337.

According to one preferred embodiment, a carbosiloxane dendrimer compound in the sense of the present invention may be conveyed in an oil or a mixture of oils, which is/are preferably volatile, chosen in particular from silicone oils and hydrocarbon-based oils, and mixtures thereof.

According to one particular embodiment, a silicone oil that is suitable for use in the invention may be cyclopentasiloxane.

According to another particular embodiment, a hydrocarbon-based oil that is suitable for use in the invention may be isododecane.

Carbosiloxane dendrimer compounds that may be particularly suitable for use in the present invention are the polymers sold under the names TIB 4-100, TIB 4-101, TIB 4-120, TIB 4-130, TIB 4-200, FA 4002 ID (TIB 4-202), TIB 4-220 and FA 4001 CM (TIB 4-230) by the company Dow Corning. Particularly preferred carbosiloxane dendrimer compounds are ACRYLATES/POLYTRIMETHYLSILOXYMETHACRYLATE COPOLYMER sold by Dow Corning under the Dowsil™ name such as commercial products FA 4002 ID and FA 4004 ID which are blends of polymer in isododecane (approximately 40% active material).

Preferably, the at least one carbosiloxane dendrimer compound is present in the compositions of the present invention in an amount ranging from about 0.5% to about 25% by weight, preferably from about 1% to about 20% by weight, preferably from about 2.5% to about 17.5% by weight, and preferably from about 3% to about 15% by weight with respect to the weight of the composition, including all ranges and subranges therebetween such as, for example, 4% to 14%, 1% to 15%, 5% to 25%, etc.

According to preferred embodiments, the at least one cyclic amide-containing polymer and the at least one carbosiloxane dendrimer compound are present in the compositions of the present invention in a weight ratio of about 3:1 to about 1:3, preferably about 2:1 to about 1:2, and preferably about 1:1.

According to preferred embodiments, the film forming component of the compositions of the invention is free of, substantially free of, or devoid of film formers other than one aqueous phase film former and the at least one carbosiloxane dendrimer compound.

### Aqueous Phase

According to preferred embodiments of the present invention, compositions comprising an aqueous phase solvent system comprising (i) water in an amount of at least about 5% by weight with respect to the total weight of the composition; and (ii) optionally, at least one C₂-C₅ monoalcohol and/or at least one polyol are provided.

According to preferred embodiments, the compositions of the present invention comprise water. Preferably, the compositions comprise at least about 5% water by weight, preferably in amounts from about 5% to about 50%, preferably from about 10% to about 45%, preferably from about 15% to about 40%, and preferably from about 20% to about 40%, by weight, based on the total weight of the composition, including all ranges and subranges therebetween such as, for example, about 15% to about 40%.

Suitable C2-C5 monoalcohols, if present, include ethanol, propanol, butanol, pentanol, isopropanol, isobutanol and isopentanol. Ethanol is particularly preferred.

Suitable polyols, if present, include diols (2 OH groups), triols (3 OH groups) and tetrols (4 OH groups) such as, for example, glycerol, propanediols, butanediols, butanetriols, pentanediols, pentanetriols, pentanetetrols, etc.

Preferably, the C2-C5 monoalcohol(s) and/or polyols, if present, is/are present in the compositions of the present invention in an amount ranging from about 1% to about 25%, preferably from about 2% to about 20%, preferably from about 3% to about 20%, and preferably from about 5% to about 20%, by weight, based on the total weight of the composition, including all ranges and subranges in between such as, for example, about 1% to about 15%.

Preferably, the aqueous phase solvent system of the compositions of the present invention consists essentially of, or consists of, water and, optionally, C2-C5 monoalcohols and/or polyols. Preferably, the aqueous phase solvent system is “free of,” “devoid of” or “substantially free of” solvents other than water and, optionally, C2-C5 monoalcohols and/or polyols.

### Oil Phase

According to the present invention, compositions comprising at least one oil are provided. Compositions of the present invention comprise sufficient oil to form a water-in-oil emulsion, preferably more than about 5% oil, preferably more than about 10% oil, preferably more than about 12% oil, preferably more than about 15% oil, preferably more than about 20% oil, and preferably more than about 25% oil, such as, for example from about 5% to about 80% oil, preferably from about 10% to about 60% oil, preferably from about 12% to about 55% oil, and preferably from about 15% to about 50% oil by weight with respect to the total weight of the composition, including all ranges and subranges therebetween.

Suitable oils include volatile and/or non-volatile oils. Such oils can be any acceptable oil including but not limited to silicone oils and/or hydrocarbon oils.

According to certain embodiments, the oil carrier comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C8 to C16 alkanes such as C8 to C16 isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |
| Undecane | 62 |
| Tridecane | 94 |
| Isohexadecane | 96 |

The volatility of the solvents/oils can be determined using the evaporation speed as set forth in U.S. Pat. No. 6,338,839, the contents of which are incorporated by reference herein.

According to other embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R⁵COOR₆ in which R⁵ represents a linear or branched higher fatty acid residue containing from 1 to 40 carbon atoms, including from 7 to 19 carbon atoms, and R⁶ represents a branched hydrocarbon-based chain containing from 1 to 40 carbon atoms, including from 3 to 20 carbon atoms, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol, cetyl alcohol, stearyl alcohol, and cetearly alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments, the compositions of the present invention are devoid of, free of, or substantially free of, silicone oils, or devoid of, free of, or substantially free of, phenylated silicone oils (as described above).

According to preferred embodiments, the compositions of the present invention are devoid of, free of, or substantially free of, waxes.

### Low HLB Surfactant

According to preferred embodiments of the present invention, compositions comprising at least one low HLB surfactant are provided. “HLB” refers to the “hydrophilic-lipophilic balance” associated with emulsifiers/surfactants. In particular, “HLB” value relates to the ratio of hydrophilic groups and lipophilic groups in emulsifiers, and also relates to solubility of the emulsifiers. Lower HLB emulsifiers are more soluble in oils (lipophilic material) and are more appropriate for use in water-in-oil (W/O) emulsions. Higher HLB emulsifiers are more soluble in water (hydrophilic material) and are more appropriate for oil-in-water (O/W) emulsions. In the context of the present invention, “low HLB surfactant” means a surfactant having an HLB value of less than or equal to 6, preferably less than 5, and preferably less than 4. Preferably, the HLB value is 3 or greater.

The low HLB value surfactant is present as all or part of a “surfactant component” in the compositions of the present invention. According to preferred embodiments of the present invention, the surfactant component has an HLB value of less than or equal to 6, preferably less than 5, and preferably less than 4. Preferably, the HLB value is 3 or greater.

According to preferred embodiments, the low HLB surfactant is nonionic.

According to preferred embodiments, the low HLB surfactant comprises a backbone and pendant group(s), wherein (1) the backbone is hydrophobic and one or more pendant groups is/are hydrophilic, or (2) the backbone is hydrophilic and one or more pendant groups is/are hydrophobic. The backbone can be silicone-based or hydrocarbon based. By “backbone,” it is meant that the surfactant comprises a main chain. By “pendant” group(s), it is meant that one or more groups is attached to the backbone or main chain of the surfactant. The pendant group(s) can be silicone-based or hydrocarbon based, and can be attached at any location along the backbone or main chain, for example at one or both terminal ends of the chain, at location(s) not at a terminal end of the chain, or both. According to preferred embodiments, the pendant group(s) include one or more ester group(s) attached to the backbone or main chain.

A preferred backbone is a hydrophobic such as, for example, one or more C8-C24 fatty compounds, preferably C12-C20 fatty compound(s), and preferably C16-C18 fatty compound(s) such as, for example, stearate, isostearate, laurate, etc, and a preferred pendant group for such a backbone is a hydrophilic group such as a polyethylene glycol (PEG) polymer or a polyglyceryl polymer.

Another preferred backbone is a hydrophilic, hydrocarbon-based backbone such as a polyethylene glycol (PEG) polymer or a polyglyceryl polymer, and a preferred pendant group for such a backbone is a hydrophobic group such as, for example, C8-C24 fatty compound(s), preferably C12-C20 fatty compound(s), and preferably C16-C18 fatty compound(s) such as, for example, stearate, isostearate, laurate, etc.

The low HLB surfactant of the present invention can be a silicone surfactant, preferably comprising dimethicone (for example, PEG-10 dimethicone or lauryl PEG-9 polydimethylsiloxyethyl dimethicone), or a non-silicone surfactant, free of dimethicone (for example, PEG-30 dipolyhydroxystearate). The low HLB surfactant of the present invention can have alkoxylation (for example, ethoxylation or propoxylation) through groups such as, for example, (C2H4O)—/polyethylene glycol groups (PEG), (C3H6O)—/polypropylene glycol groups (PPG), etc. Suitable emulsifiers include alkoxylated compounds, partial glycerides of alkoxylated compounds, polyglycerolated compounds, and mixtures thereof, etc. Polyglycerolated compounds are preferred.

Suitable polyglycerolated compounds include, but are not limited to, compounds preferably containing 2 to 20 glyceryl groups, preferably 3 to 15 glyceryl groups, and preferably 4 to 10 glyceryl groups. Polyglyceryl-4 Diisostearate/Polyhydroxystearate/Sebacate, sold under the tradename Isolan GPS by Evonik, is particularly preferred.

The at least one low HLB surfactant is preferably present in the compositions of the present invention in an amount of about 1% to about 15%, preferably from about 1.5% to about 10%, and preferably from about 2% to about 8% by weight with respect to the total weight of the composition, including all ranges and subranges therebetween.

### Colorant (Coloring Agent)

According to the present invention, compositions comprising at least one coloring agent are provided. According to preferred embodiments, the at least one coloring agent is at least one surface-treated pigment. “Surface-treated pigment” means pigments that have totally or partially undergone a surface treatment of chemical, electronic, electrochemical, mechanochemical or mechanical nature, with a surface treatment agent. Preferably, the pigments are selected from inorganic pigments or inorganic/organic mixed pigments.

Surface treatment agents may be selected from the group consisting of alkyl silanes, organotitanates, halogenated phosphonates, and halogenated organosilanes. According to preferred embodiments, pigments have been surface treated with a surface treatment agent selected from the group consisting of alkoxylated alkyl silanes such as, for example, ethoxylated and/or propoxylated C2-C8 alkyl silanes, and salts thereof, organotitanates such as, for example, titanium salts of fatty acids such as, for example, C2-C8 alkylated titanium salts of C9-C24 fatty acids such as stearic acid, isostearic acid, oleic acid, cetearic acid, cetyl acid, etc., halogenated organophosphonates such as, for example, perfluoroalkyl phosphonates, and salts thereof, and halogenated organosilanes such as, for example, perfluoro C2-C8 alkyl silanes (optionally ethoxylated and/or propoxylated), and salts thereof. Specific examples of suitable surface treatment agents include (1) triethoxy caprylylsilane, (2) perfluorooctyltriethoxysilane, (3) sodium perfluorohexylethylphosphonate and (4) isopropyl titanium triisosterate, (5) triethoxysilylethylpolydimethylsiloxyethyl dimethicone, and (6) combinations thereof.

The surface-treated pigments of the present invention can be prepared according to surface treatment techniques well known to a person of ordinary skill in the art or can be found commercially.

For example, the surface treatment agent with which the pigments are treated can be deposited on the pigments by solvent evaporation, chemical reaction between the molecules of the surface treatment agent or creation of a covalent bond between the surface treatment agent and the pigments. The surface treatment can thus be carried out, for example, by chemical reaction of a surface treatment agent with the surface of the pigments and creation of a covalent bond between the surface treatment agent and the pigments. An exemplary method is described, for example, in U.S. Pat. No. 4,578,266, the entire contents of which is hereby incorporated by reference.

The at least one surface-treated pigment, if present, preferably is present in the compositions of the present invention in an active solid content amount ranging from about 1% to about 30%, preferably from about 3% to about 25%, and preferably from about 5% to about 20%, by weight with respect to the total weight of the composition, including all ranges and subranges there between.

According to preferred embodiments of the present invention, compositions further comprising at least one unsurfaced-treated (not surface-treated) coloring agent are provided. Such coloring agents, if present, may be in addition to, or instead of, the surface-treated pigment discussed above.

According to this embodiment the coloring agent is preferably chosen from pigments which are not surface-treated, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 40% by weight of the total weight of the composition, such as from 0.0001% to 30%, including all ranges and subranges therebetween.

The nacreous pigments which may be used according to the present invention may be chosen from colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.0001% to 40%, preferably from 0.001% to 30%, including all ranges and subranges therebetween.

The non-surface treated pigments, which may be used according to the present invention, may be chosen from white, colored, inorganic, organic, polymeric, and nonpolymeric pigments. Representative examples of mineral pigments include titanium dioxide, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

The coloring agents may be present in the composition in a concentration ranging up to 50% by weight of the total weight of the composition, such as from 0.0001% to 40%, and further such as from 0.001% to 30%, including all ranges and subranges therebetween.

### Thickening Agent/Colorant/Active Agent

### Thickening Agent

According to preferred embodiments of the present invention, compositions further comprising at least one thickening agent are provided. Suitable thickening agents include agents for thickening an oil phase of a composition, agents for thickening a water phase of a composition, and mixtures thereof.

According to preferred embodiments, the thickening agent is a gelling agent for gelling water (water phase) in the composition. Suitable gelling agents include any gelling agent, such as for example, polysaccharide gums, clays and acrylic acid (co) polymers, such as high molecular weight homo- or co-polymers comprising acrylic acid, optionally crosslinked with a polyalkenyl polyether, including some polymers identified as “carbomer” as well as amphiphilic polymers. Preferably, the gelling agent is an amphiphilic polymer or a clay, or a combination thereof.

According to preferred embodiments, the gelling agent(s) may be at least one clay. Among the clays, mention may be made of clays of the smectite family, such as laponite, of the kaolinite family, such as kaolinite, dickite, nacrite, optionally modified clays of the halloysite, dombassite, antigorite, berthierine, pyrophyllite, montmorillonite, beidellite, vermiculite, talc, stevensite, hectorite, saponite, chlorite, sepiolite and illite family.

Clays are products that are already well known per se, which are described, for example, in the publication “Mineralogie des argiles” [“Clay Mineralogy”], S. Caillère, S. Henin, M. Rautureau, 2nd Edition 1982, Masson, the teaching of which is included herein by way of reference.

Natural clay is a sedimentary rock composed to a large extent of specific minerals, silicates generally of aluminium. Kaolin is thus a natural clay.

The clays may also be synthetic. Thus, Sumecton mentioned below is a synthetic saponite.

The clays may also be chemically modified with various compounds, such as acrylic acids, polysaccharides (for example carboxymethylcellulose) or organic cations.

Preferably, in the context of the present invention, use is made of clays that are cosmetically compatible and acceptable with keratin materials. Clays that may especially be mentioned include kaolinite, montmorillonites, saponites, laponites, hectorites, and illites. Mixtures of clays and natural clays may also be used.

Natural clays that may be mentioned include green clays, in particular rich in illite; clays rich in montmorillonite, known as fuller's earth, or such as bentonite or else white clays rich in kaolinite. As bentonites, or modified bentonites, mention may in particular be made of those sold under the names Bentone 38 VCG®, Bentone Gel CAO V®, Bentone 27 V®, Bentone Gel ISD V, and Bentone Gel MIO VR by the company Elementis.

By way of clay rich in montmorillonite, mention may be made of the aluminium silicate hydrate sold under the name Gel White H@ by the company Rockwood.

By way of saponite, which belongs to the montmorillonite family, mention may be made of synthetic saponite, in particular the product sold by the company Kunimine under the name Sumecton®.

By way of tack, mention may be made of those sold under the names Rose Talc® and Talc SG-2000@ sold by the company Nippon Talc, Luzenac Pharma MR sold by the company Luzenac, J-68BC® from US Corporation and Micro ACE-P-3@ sold by the company Nippon Talc.

According to preferred embodiments, the gelling agent(s) may be at least one polysaccharide gum. Among the polysaccharide gums, mention may be made of polysaccharide gums may be chosen from polysaccharide gums produced by microorganisms; polysaccharide gums isolated from algae, and polysaccharide gums from higher plants, such as homogeneous polysaccharide gums, in particular celluloses, carrageenans, gellan gum, agar-agar, xanthan gum, alginate-based compounds, in particular sodium alginate, scleroglucan gum, guar gum, etc. In general, the compounds of this type that may be used in the present invention are chosen from those described especially in Kirk-Othmer's Encyclopedia of Chemical Technology, Third Edition, 1982, volume 3, pp. 896-900, and volume 15, pp. 439-458, in Polymers in Nature by E. A. McGregor and C. T. Greenwood, published by John Wiley & Sons, Chapter 6, pp. 240-328, 1980, in the publication by Robert L. Davidson entitled Handbook of Water-soluble Gums and Resins published by McGraw-Hill Book Company (1980) and in Industrial Gums-Polysaccharides and their Derivatives, edited by Roy L. Whistler, Second Edition, published by Academic Press Inc.

Preferably, if present, the thickening agent(s) is/are present in the compositions of the present invention in amounts ranging from about 0.1 to about 30% by weight, preferably from 0.5 to 25% by weight, preferably from 1 to 20% and preferably from 2.5 to 15% by weight, all weights based on the weight of the composition as a whole, including all ranges and subranges therebetween such as, for example, 0.1 to 1.5%, 2 to 20%, 10 to 20%, etc. Active Agent

According to preferred embodiments of the present invention, compositions further comprising at least one active agent are provided. Preferably, the active agent is in the aqueous phase (hydrophilic active agent), although active agents may be present in the oil phase (hydrophobic active agent).

In some embodiments, the active agent may be:
a moisturizing agent, such as a polyol such as, for example, glycerin and sugars, urea and its derivatives, such as in particular hydroxyalkyl urea, in particular hydroxyalkylurea, and mixtures thereof;
a desquamating agent, which may be a compound capable of acting either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives; or acting on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and in particular (N-2-hydroxyethylpiperazine-N-2-ethane) sulfonic acid (HEPES); 2-oxothiazolidine-4-carboxylic acid (procysteine) derivatives; α-amino acid derivatives of the type such as glycine (as described in EP-0 852 949 and sodium methylglycinediacetate sold by BASF under the trade name Trilon M); honey; sugar derivatives such as O-octanoyl-6-D-maltose and N-acetylglucosamine;
an amino acid, which may include, for example, lysine, glycine, sarcosine, arginine, histidine, leucine, etc.;
a humectant;
an anti-aging agent, which may include, for example, one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof.
a mattifying agent, which may include, but is not limited to, mattifying fillers such as, for example, talc, silica, silicone elastomers, and polyamides, and waxes such as, for example, beeswax and copernicia cerifera (carnauba) wax.
an antimicrobial agent, non-limiting examples of which include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in patent WO 93/18743, farnesol and phytosphingosines, and mixtures thereof;
a pigment modifying agent and/or skin lightening agents, such as double-stranded RNA oligonucleotides are useful for decreasing tyrosinase expression. Mention may also be made of ceramides, vitamin C and derivatives thereof, in particular vitamin CG, CP and 3-O ethyl vitamin C, alpha- and beta-arbutin, ferulic acid, kojic acid, resorcinol and derivatives thereof, calcium D-pantetheine sulphonate, lipoic acid, ellagic acid, vitamin B3, phenylethyl resorcinol, for instance Symwhite 377® from the company Symrise, a kiwi fruit (Actinidia chinensis) juice sold by Gattefosse, an extract of Paeonia suffructicosa root, such as the product sold by the company Ichimaru Pharcos under the name Botanpi Liquid BR, an extract of brown sugar (Saccharum officinarum), such as the extract of molasses sold by the company Taiyo Kagaku under the name Molasses Liquid, a mixture of undecylenic acid and undecylenoyl phenyl alanine, such as Sepiwhite MSH® from Seppic;
a vitamin such as a vitamin A compound such as retinol or retinoic acid, vitamin B compound such as vitamin B1, B3 or B6, in particular niacinamide, a vitamin C compound such as an ascrobyl phosphate salt (e.g., magnesium or sodium, and/or derivatives thereof;
an ultraviolet (UV) filter such as, e.g., an aminobenzoic acid derivative, a dibenzoylmethane derivative, a salicylic acid derivative, a cinnamic derivative, a β,β diphenylacrylate derivative, a benzophenone derivative, benzylidene camphor derivative, and mixtures thereof. Mention may be made especially of ethylhexyl methoxycinnamate sold under the tradename UVINUL MC 80@ by the company BASF, of ethylhexyl salicylate sold under the tradename NEO HELIOPAN OS® by the company SYMRISE and of octocrylene sold under the tradename NEO HELIOPAN 303@ by the company SYMRISE; and/or
a combination thereof.

### Additional Additives

The composition of the invention can also comprise any additive usually used in the field under consideration. For example, additional film forming agents (in addition to carbosiloxane dendrimer compound and the at least one aqueous phase film former), waxes, dispersants such as poly(12-hydroxystearic acid), preserving agents, fragrances, fillers, antioxidants, neutralizing agents, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002). However, it is to be understood that preferred embodiments of the present invention include compositions which are “free,” substantially free” or “devoid” of the ingredients discussed in this paragraph such as additional film forming agents and waxes as previously-discussed as well as fillers, in particular mattifying fillers (compositions are “free of mattifying fillers,” “substantially free of mattifying fillers,” or “devoid of mattifying fillers.”

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to the eyelashes of human beings.

According to preferred embodiments, methods of treating, caring for and/or enhancing the appearance of skin by applying compositions of the present invention to the skin in an amount sufficient to treat, care for and/or enhance the appearance of the skin are provided. In accordance with these preceding preferred embodiments, the compositions of the present invention are applied topically to the desired area of the skin in an amount sufficient to treat, care for and/or enhance the appearance of the skin. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to preferred embodiments of the present invention, compositions having improved cosmetic properties such as, for example, increased water-resistance, sebum-resistance and/or transfer-resistance are provided. Preferably, the composition is a composition for skin. Most preferably, the composition is a foundation further comprising at least one colorant, preferably a surface-treated pigment.

According to preferred embodiments of the present invention, compositions having “matte” properties are provided, in the presence or absence of sebum testing as discussed herein.

In the absence of sebum, compositions can have, for example, average gloss properties, measured at 60° of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc.

In the absence of sebum, compositions can have, for example, average gloss properties, measured at 85° of less than or equal to 20, for example 19, preferably less than or equal to 18, 16, 15, 14 or 10, including all ranges and subranges therebetween such as 10-20, 10-15, 12-20, 13-18, less than 11, less than 13, less than 15, less than 17, etc.

In the presence of sebum, compositions can have, for example, average gloss properties, measured at 60° of less than or equal to 15, for example 14, preferably less than or equal to 13, 12, 11, 10, 9, 8, 6, 5, 4 or 1, including all ranges and subranges therebetween such as 10-15, 6-15, 6-10, 3-8, less than 10, less than 13, less than 5, etc.

In the presence of sebum, compositions can have, for example, average gloss properties, measured at 85° of less than or equal to 25, for example 23, preferably less than or equal to 22, 21, 20, 18, 16, 15, 14 or 10, including all ranges and subranges therebetween such as 10-25, 20-25, 12-20, 18-25, less than 21, less than 23, less than 15, less than 17, etc.

According to referred embodiments of the present invention, methods of making a water-in-oil (w/o) emulsion composition comprising an aqueous phase dispersed in a continuous oil phase, comprising combining (adding) at least one carbosiloxane dendrimer compound in an oil phase and at least one aqueous phase film former in an aqueous phase during formation of the emulsion composition. Preferably, the composition is a composition for skin. Preferably, the at least one carbosiloxane dendrimer compound and at least one aqueous phase film former are combined or added in the amounts and/or ratios discussed above. Most preferably, the composition is a foundation comprising at least one colorant, preferably a surface-treated pigment.

According to preferred embodiments of the present invention, kits comprising, as separate compositions in one or more containers within the kits, (A) a composition of the present invention as discussed above, in particular a foundation for application to skin, comprising at least one carbosiloxane dendrimer compound and at least one aqueous phase film former and (B) at least one other composition such as a primer coat composition, a basecoat composition, or a topcoat composition are provided. Compositions (A) and (B) may be contained in different portions or sections of the same container within the kit. However, compositions (A) and (B) may also be in different containers with the kit.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Foundation Composition

| | Total 100%- | Total 100%- |
|---|---|---|
| | General | Preferred |
| Chemical Name/Function | % wt/wt | % wt/wt |
| Surfactants | 1-10% | 1-5% |
| Aqueous Phase Film Former | 0.5-10% | 1-5% |
| Carbosiloxane dendrimer | 0.5-10% | 1-5% |
| compound | | |
| Oils | 5-50% | 20-40% |
| Colorants | 1-30% | 5-20% |
| Water | QS | QS |
| Monoalcohol | 1-20% | 3-10% |
| Polyol | 1-10% | 2-8% |
| Active Agent (e.g. vitamin) | 1-20% | 5-20% |
| Clays | 0.1-10% | 0.1-5% |
| Fillers | 0.5-10% | 1-5% |

### Example 2—Comparative Composition A and Invention Composition B; Matte and Transfer Properties Testing

The following compositions were prepared:

| | Comparative | Invention |
|---|---|---|
| INCI Name | Composition A | Composition B |
| WATER | 35.67 | 21.18 |
| Additional Additives | 10.44 | 10.44 |
| Cellulose Thickener | 0.26 | 0.26 |
| VP/METHACRYLAMIDE/VINYL | — | 14.49 |
| IMIDAZOLE COPOLYMER | | |
| (Luviset Clear AT3-20% | | |
| active polymer in water) | | |
| ALCOHOL DENAT. | 4.35 | 4.35 |
| Polyglyceryl Surfactant | 2.61 | 2.61 |
| Volatile Hydrocarbon Oil | QS | QS |
| Clay Thickener | 1.31 | 1.31 |
| Propylene Carbonate | 0.39 | 0.39 |
| Filler Particles | 0.44 | 0.44 |
| ACRYLATES/POLYTRIMETHYL- | 8.7 | 8.7 |
| SILOXYMETHACRYLATE | | |
| COPOLYMER | | |
| (DOWSIL FA4004 ID Silicone | | |
| Acrylate-40% active polymer in | | |
| Isododecane) | | |
| HYDROPHOBICALLY | 13 | 13 |
| MODIFIED PIGMENTS | | |

### Example 2A: Resistance to Gloss in the Presence of Sebum Measure at 60°

The prepared compositions were applied to an opacity card using a 6 mil drawdown bar. Compositions were then mixed with artificial sebum by combining 95% composition with 5% of artificial sebum to make up 100% total. This was then applied using a 6 mil drawdown bar to an opacity card. All films were dried at approximately 37° C. and gloss values were read using a BYK micro-TRI-gloss meter at 60° angle.

It was observed that the Invention Composition B containing VP/METHACRYLAMIDE/VINYL IMIDAZOLE COPOLYMER (Luviset Clear AT3) was initially less glossy, and maintained a more matte appearance in the presence of artificial sebum.

| | Gloss value (60°) | Gloss Value (60°) + 5% Sebum |
|---|---|---|
| Composition A | 7.7 | 34.9 |
| Composition B | 5.5 | 10.7 |

### Example 2B: Resistance to Gloss in the Presence of Sebum Measure at 85°

The prepared compositions were applied to an opacity card using a 6 mil drawdown bar. Compositions were then mixed with artificial sebum by combining 95% composition with 5% of artificial sebum to make up 100% total. This was then applied using a 6 mil drawdown bar to an opacity card. All films were dried at approximately 37° C. and gloss values were read using a BYK micro-TRI-gloss meter at 85° angle.

It was observed that the Invention Composition B containing VP/METHACRYLAMIDE/VINYL IMIDAZOLE COPOLYMER (Luviset Clear AT3) was initially less glossy, and maintained a more matte appearance in the presence of artificial sebum.

| | Gloss Value | Gloss value (85°) + 5% |
|---|---|---|
| | (85º) | Sebum |
| Composition A | 20.6 | 44.1 |
| Composition B | 16 | 20.9 |

### Example 2C: Resistance to Transfer

20 microliters of compositions A and B each were applied to a forearm in a square of equivalent area. The compositions were allowed to dry for 45 minutes. Each composition was then wiped using a cotton cloth (ISO 105 F09 Crock Square).

It was observed that both formulations exhibited transfer resistance, but Invention Composition B which contained VP/METHACRYLAMIDE/VINYL IMIDAZOLE COPOLYMER and ACRYLATES/POLYTRIMETHYLSILOXYMETHACRYLATE COPOLYMER (DOWSIL FA4004 ID Silicone Acrylate) had enhanced resistance to transfer of color to the cotton cloth.

### Example 3: Compositions C—F and Testing

The following compositions were prepared:

| | Com- | | Com- | |
|---|---|---|---|---|
| | parative | Invention | parative | Invention |
| | Com- | Com- | Com- | Com- |
| | position | position | position | position |
| Composition | C | D | E | F |
| INCI | | | | |
| WATER | 35.11 | 20.46 | 35.11 | 20.46 |
| ADDITIONAL ADDITIVES | 10.65 | 10.65 | 10.65 | 10.65 |
| CELLULOSE THICKENER | 0.26 | 0.26 | 0.26 | 0.26 |
| VP/METHACRYLAMIDE/ | — | 14.65 | — | 14.65 |
| VINYL IMIDAZOLE | | | | |
| COPOLYMER | | | | |
| (LUVISET CLEAR AT3-20% | | | | |
| ACTIVE POLYMER IN | | | | |
| WATER) | | | | |
| ALCOHOL DENAT. | 4.40 | 4.40 | 4.40 | 4.40 |
| POLYGLYCERYL | 2.64 | 2.64 | 2.64 | 2.64 |
| SURFACTANT | | | | |
| CLAY THICKENER | 1.32 | 1.32 | 1.32 | 1.32 |
| PROPYLENE CARBONATE | 0.40 | 0.40 | 0.40 | 0.40 |
| VOLATILE | QS | QS | QS | QS |
| HYDROCARBON OIL | | | | |
| FILLERS AND EFFECT | 2.38 | 2.38 | 2.38 | 2.38 |
| PARTICLES | | | | |
| ACRYLATES/ | 8.8 | 8.8 | 0 | 0 |
| POLYTRIMETHYL- | | | | |
| SILOXYMETHACRYLATE | | | | |
| COPOLYMER | | | | |
| (DOWSIL FA4004 ID | | | | |
| SILICONE ACRYLATE-40% | | | | |
| ACTIVE POLYMER | | | | |
| IN ISODODECANE) | | | | |
| ACRYLATES/ | 0 | 0 | 8.8 | 8.8 |
| POLYTRIMETHYL- | | | | |
| SILOXYMETHACRYLATE | | | | |
| COPOLYMER | | | | |
| (DOWSIL FA4002 ID | | | | |
| SILICONE ACRYLATE-40% | | | | |
| ACTIVE POLYMER | | | | |
| IN ISODODECANE) | | | | |
| HYDROPHOBICALLY | 12 | 12 | 12 | 12 |
| MODIFIED PIGMENTS | | | | |

### Example 3A: Resistance to Removal and Transfer in the Presence of Sebum

20 μL of formulations C, D, E and F were applied to white artificial leather substrate. Applied formulations were then dried at 37° C. overnight. The color of each applied formulation in L*A*B* color space was determined using a datacolor spectrophotometer.

Formulations were then wiped with an artificial sebum-soaked cloth (ISO 105 F₀₉ Crock Squares) in two controlled motions with alternating direction and controlled force by placing the protein leather on a pressure sensor. Samples were then allowed to dry and LAB readings were taken again. The normalized ΔL was then calculated as:

$$Normalized Δ L = \frac{ABS ( {L}_{final} - {L}_{initial} )}{ABS ( {L}_{Substrate} - {L}_{initial} )} \times 100$$

Where ABS is absolute value, L_{final} is final L value in LAB color space of the deposited formula, Lᵢₙᵢₜᵢₐₗ is the initial L value in LAB color space of the deposited formula, L_{substrate} is the L value of the substrate. Higher values of Normalized ΔL correspond to a larger change in color of the deposited formulation after wiping, which indicates less resistance to removal and transfer in the presence of artificial sebum. All samples were tested in triplicate and the data was presented as average of Normalized ΔL and standard deviation of the Normalized ΔL values.

It was observed that both Invention Compositions D and F which contained VP/METHACRYLAMIDE/VINYL IMIDAZOLE COPOLYMER (Luviset Clear AT3) had lower values of Normalized ΔL, indicating increased resistance to removal and transfer in the presence of artificial sebum.

| Composition | AVG Normalized AL | STDEV Normalized AL |
|---|---|---|
| Comparative | 18.22 | 7.93 |
| C | | |
| Invention | 5.51 | 0.78 |
| D | | |
| Comparative | 16.30 | 2.13 |
| E | | |
| Invention | 3.46 | 1.16 |
| F | | |

What is claimed is:

1. A water-in-oil emulsion composition comprising an aqueous phase dispersed in a continuous oil phase, comprising at least one carbosiloxane dendrimer compound in the oil phase, at least one aqueous phase film former in the aqueous phase, at least one oil, at least one colorant and water.

2. The composition of claim 1, wherein the composition further comprises at least one thickening agent and/or at least one active agent.

3. The composition of claim 1, wherein the composition further comprises at least one thickening agent and at least one active agent.

4. The composition of claim 2, wherein at least one thickening agent is present in the composition, and the at least one thickening agent is a clay, preferably a hectorite.

5. The composition of claim 1, wherein the at least one coloring agent is a surface-treated pigment.

6. The composition of claim 2, wherein at least one active agent is present in the composition, and the at least one active agent is a vitamin, preferably a vitamin B compound, preferably niacinamide.

7. The composition of claim 1, wherein the at least one carbosiloxane dendrimer compound is a silicone-modified carbosiloxane dendrimer compound, preferably ACRYLATES/POLYTRIMETHYLSILOXYMETHACRYLATE COPOLYMER.

8. The composition of claim 1, wherein the at least one aqueous phase film former is at least one cyclic amide-containing polymer which comprises a pyrrolidone monomer, a cyclic amine monomer, and an acrylamide monomer copolymer, and preferably is a copolymer of N-vinyl pyrrolidone, methacrylamide, and N-vinylimidazole.

9. The composition of claim 1, wherein the at least one aqueous phase film former is at least one homopolymer of vinylpyrrolidone, at least one copolymer comprising vinylpyrrolidone, or combinations thereof.

10. The composition of claim 1, wherein the weight ratio of aqueous phase film former to carbosiloxane dendrimer compound is from about 3:1 to about 1:3, preferably about 1:1.

11. The composition of claim 1, wherein the composition has average gloss properties, measured at 60° in the presence of sebum, of less than or equal to 15.

12. The composition of claim 1, wherein the composition is substantially free of phenylated silicone oil.

13. The composition of claim 1, wherein the composition comprises at least about 12% by weight of oil with respect to the weight of the composition.

14. A method of making up skin comprising applying the composition of claim 1 to skin in an amount sufficient to make up the skin.
